(19) United States
(12) Patent Application Publication
(10) Pub. No.: US 2026/0078128 A1
(43) Pub. Date: Mar. 19, 2026

(54) Process for the Preparation of Ruxolitinib and Novel Crystalline Form Thereof

(71) Applicant: Granules India Limited, Hyderabad (IN)

(72) Inventors: Venkata Srinivas Pullela, Hyderabad (IN); Sampath Kumar Upparapalli, Hyderabad (IN); Venkateswara Rao Kalapala, Hyderabad (IN); Chennakesava Reddy Bandi, Hyderabad (IN); Basaveswara Rao Bavireddi, Hyderabad (IN); Chakradhar Gadde, Hyderabad (IN); Prasad Ravi, Hyderabad (IN)

(21) Appl. No.: 19/336,597

(22) Filed: Sep. 23, 2025

Related U.S. Application Data
(63) Continuation of application No. PCT/IB2024/057165, filed on Jul. 24, 2024.

(30) Foreign Application Priority Data
Sep. 4, 2023 (IN) 202341059245
Feb. 12, 2024 (IN) 202441009264
Mar. 12, 2024 (IN) 202441017545

(51) Int. Cl. C07D 487/04

(52) U.S. Cl. CPC C07D 487/04

(57) ABSTRACT

The present invention relates to an improved process for the preparation of ruxolitinib or its salts and intermediates thereof. The present invention also relates to a novel crystalline form G of ruxolitinib, a process for its preparation, use of it in the preparation of ruxolitinib salts, a pharmaceutical composition comprising it, and method of use thereof. The present invention also relates to an improved process for preparation of crystal modification 3 of ruxolitinib hydrochloride salt.

### PRIORITY

This application claims the benefit under Indian Provisional Application No(s). 202341059245 filed on Sep. 4, 2023, entitled “IMPROVED PROCESS FOR THE PREPARATION OF RUXOLITINIB”, 202441009264 filed on Feb. 12, 2024, entitled “NOVEL CRYSTALLINE FORM OF RUXOLITINIB”, and 202441017545 filed on Mar. 12, 2024, entitled “NOVEL CRYSTALLINE FORM OF RUXOLITINIB AND PROCESS FOR THE PREPARATION THEREOF” the content of each of which are incorporated by reference herein.

### FIELD OF INVENTION

The present invention relates to an improved process for the preparation of ruxolitinib or its salts and intermediates thereof. The present invention also relates to a novel crystalline form G of ruxolitinib, a process for its preparation, use of it in the preparation of ruxolitinib salts, a pharmaceutical composition comprising it, and method of use thereof. The present invention also relates to an improved process for preparation of crystal modification 3 of ruxolitinib hydrochloride salt.

### BACKGROUND OF THE INVENTION

Ruxolitinib is a selective JAK1/JAK2 tyrosine kinase inhibitor developed by Incyte Corporation and Novartis Corporation. It is the first drug (Trade name: Jakafi) approved by the US FDA in November 2011 for the treatment of myelofibrosis, and indications thereof are intermediate or high-risk myelofibrosis, including primary myelofibrosis, secondary polycythemia myelofibrosis and post-essential thrombocythemia myelofibrosis.

Ruxolitinib chemically known as (R)-3-(4-(7H-pyrrolo[2,3-d]pyrimidin-4-yl)-1H-pyrazol-1-yl)-3-cyclopentylpropanenitrile and has the following structural formula I:

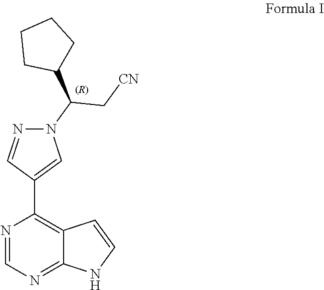

The U.S. Pat. No. 7,598,257 patent discloses ruxolitinib, its pharmaceutically acceptable salts thereof. Also, it discloses a process for the preparation of Ruxolitinib which involves using HPLC methods to separate intermediates, also, involves in the use of column chromatography purification methods for the final product, which is laborious and time consuming and suffers from poor yield and low purity.

The U.S. Pat. No. 8,410,265 patent discloses a process for the preparation of ruxolitinib, involves a chemical resolution of D-(+)-dibenzoyltartaric acid, asymmetric hydrogenation under the action of a chiral metal catalyst and a chiral small molecule catalyzed asymmetric synthesis route of the ruxolitinib. Wherein, the chemical resolution and repeated recrystallization are carried out, and the yield is low; the starting materials for asymmetric hydrogenation are not easy to prepare, and the chiral catalyst is difficult to prepare; the chiral small molecular catalyst is a non-commercial product, the preparation cost is high, and the stereoselectivity of the reaction is not high.

Above prior-art methods involve chiral resolution reactions at final stage which leads yield loss, chromatographic purification methods and the use of costly chiral and hazardous reagents. Using complex chiral agents increases the cost of production. Usage of chromatographic purification methods not suitable for industrial production.

Still there is a need in the art for an improved process for the preparation of ruxolitinib or salts thereof which is economical, easy to scale up and commercially viable. Thus, the main objective of the present invention is to provide an improved process for the preparation of ruxolitinib or salts thereof which avoids the shortcomings of the prior art.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predictable solubility profiles. It is desirable to investigate all solid state forms of a drug, including all polymorphic forms and solvates, and to determine the stability, dissolution and flow properties of each polymorphic form.

Ruxolitinib is one of the important pharmaceutical products approved in the US & Europe as an oral medication for treatment of myelofibrosis, polycythemia vera, acute graft versus host disease, and chronic graft versus host disease. Ruxolitinib has also been approved as a topical cream for treatment of atopic dermatitis and vitiligo. The development of new polymorphic form of a pharmaceutically useful compound provides a new opportunity to improve the performance characteristics of a pharmaceutical product such as ruxolitinib.

WO2017008772A1 describes preparation of crystal modification 3 of ruxolitinib hydrochloride salt. But the prior art processes do not produce crystal modification 3 of ruxolitinib hydrochloride salt in a single step reaction i.e., reaction of ruxolitinib with hydrochloric acid step. They involve multiple steps, including first isolating crystal modification 1, converting crystal modification 1 to crystal modification 2, and then converting crystal modification 2 to crystal modification 3. It is evident that these prior art processes are very laborious and commercially not feasible.

There still remains a need for development of new polymorphic forms of ruxolitinib and improved process for preparation thereof. The present inventors have developed novel crystalline forms of ruxolitinib and its process for preparation.

### OBJECTS OF THE INVENTION

It is an object of the present invention to overcome the shortcomings of the prior art.

It is an object of the present invention to provide an improved process for the preparation of ruxolitinib or a salt thereof, which is economical, easy to scale up and commercially viable.

It is another object of the present invention to provide an improved process for the preparation of intermediate compounds of ruxolitinib.

It is another object of the present invention to provide novel crystalline forms of ruxolitinib, a process for its preparation, use of it in the preparation of ruxolitinib salts, a pharmaceutical composition comprising it, and its use for the treatment of a JAK-associated disease or disorder in an individual.

It is yet another object of the present invention to provide an improved process for preparation of crystal modification 3 of ruxolitinib hydrochloride salt.

### SUMMARY OF THE INVENTION

According to an aspect of the present invention, there is provided an improved process for the preparation of ruxolitinib or a salt thereof, which is economical, easy to scale up and commercially viable.

According to another aspect of the present invention, there is provided an improved process for preparing intermediates of ruxolitinib.

According to another aspect of the present invention, there is provided an improved process for the preparation of intermediate compound of formula 3, in the presence of a reagent prepared in-situ from triflic anhydride and DMF and a suitable solvent and use of the intermediate formula 3 in the preparation of ruxolitinib or salt thereof.

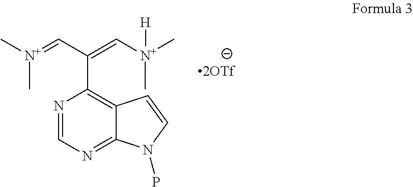

According to yet another aspect of the present invention, there is provided a novel crystalline form G of ruxolitinib, a process for its preparation, use of it in the preparation of ruxolitinib salts, a pharmaceutical composition comprising it, and its use for the treatment of a JAK-associated disease or disorder in an individual.

According to another aspect of the present invention, there is provided a novel crystalline Form G of ruxolitinib characterized by its PXRD pattern having one or more peaks at about 10.1, 13.6, 17.5 and 22.8±0.2° 2θ.

According to yet another aspect of the present invention, there is provided a novel crystalline Form G of ruxolitinib characterized by a DSC thermogram having exothermic peak at about 138-146° C.

According to another aspect of the present invention, there is provided a process for the preparation of novel crystalline Form G.

According to another aspect of the present invention, there is provided the use of crystalline Form G of ruxolitinib in the preparation of ruxolitinib salts.

According to another aspect of the present invention, there is provided an improved process for the preparation of crystalline ruxolitinib hydrochloride salt.

According to yet another aspect of the present invention, there is provided a pharmaceutical composition comprising novel crystalline form G of ruxolitinib and one or more pharmaceutically acceptable carriers, diluents, or excipients.

According to yet another aspect of the present invention, there is provided use of a novel crystalline form G of ruxolitinib for the treatment of a JAK-associated disease or disorder.

According to yet another aspect of the present invention, there is provided an improved process for the preparation of crystal modification 3 of ruxolitinib hydrochloride salt,

### BRIEF DESCRIPTION OF THE DRAWINGS

FIG. 1: X-Ray Powder Diffraction (XRPD) pattern of the crystalline form G of ruxolitinib.

FIG. 2: Differential Scanning calorimetry (DSC) thermogram of the crystalline form G of ruxolitinib.

FIG. 3: Thermogravimetric Analysis (TGA) of the crystalline form G of ruxolitinib.

FIG. 4: X-Ray Powder Diffraction (XRPD) pattern of the crystal modification 3 of ruxolitinib hydrochloride.

### DETAILED DESCRIPTION

The following description with accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the scope of the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, steps or components but does not preclude the presence or addition of one or more other features, steps, components or groups thereof.

The term “base” used herein the present invention until unless specified is selected from inorganic bases like “alkali metal hydroxides” such as lithium hydroxide, sodium hydroxide, potassium hydroxide and the like; “alkali metal carbonates” such sodium carbonate, potassium carbonate, lithium carbonate and the like; “alkali metal bicarbonates” such as sodium bicarbonate, potassium bicarbonate, lithium bicarbonate and the like; “alkali metal hydrides” such as sodium hydride, potassium hydride, lithium hydride and the like; “alkali metal alkoxides” such as sodium methoxide, sodium ethoxide, sodium tert-butoxide, potassium methoxide, potassium ethoxide, potassium tert-butoxide and the like, ammonia and organic bases such as triethylamine, methylamine, ethylamine, 1,8-diazabicycle[5.4.0]undec7-ene (DBU), 1,5-diazabicyclo(4.3.0)non-5-ene (DBN), lithiumdiisopropyl-amine (LDA), n-butyl lithium, tribenzylamine, isopropyl amine, diisopropylamine (DIPA), diisopropylethyl amine (DIPEA), N-methylmorpholine (NMM), N-ethylmorpholine, piperidine, dimethyl amino pyridine (DMAP), morpholine, pyridine, 2,6-lutidine, 2,4,6-collidine, imidazole, 1-methylimidazole, 1,2,4-triazole, 1,4-diazabicyclo[2.2.2]octane (DABCO) and the like or mixtures thereof.

The term “solvent/suitable solvent” used in the present invention is selected from the group comprising of water, alcohols, ethers, amides, esters, nitriles, sulfoxides, ketones, hydrocarbons and halogenated solvents; wherein alcohol is selected from the group consisting of methanol, ethanol, iso-propanol (IPA), n-butanol, iso-butanol and the like; ester is selected from the group consisting of ethyl acetate, isopropyl acetate (IPAc) and the like; ketone is selected from the group consisting of acetone, methyl isobutyl ketone, methyl ethyl ketone and the like; ether is selected from the group consisting of methyl tert-butyl ether, diisopropyl ether, diethyl ether tetrahydrofuran, 2-methyl tetrahydrofuran, cyclopentyl methyl ether, dioxane and the like; halogenated solvent is selected from the group consisting of dichloromethane, chloroform, chlorobenzene, bromobenzene and the like; hydrocarbons is selected from the group consisting of heptane, hexane, cyclohexane, cycloheptane, toluene, xylene, cyclohexane and the like; nitrile is selected from the group consisting of acetonitrile (ACN), propionitrile and the like; amide is selected from the group consisting of N,N-dimethylformamide (DMF), N,N-dimethyl acetamide (DMAc) and the like; sulfoxide such as dimethyl sulfoxide; sulfone; or mixtures thereof.

Suitable “amino protecting group ‘P’ can be selected from but not limited to benzyloxycarbonyl (Cbz), 2,2,2-trichloroethoxycarbonyl (Troc), 2-(trimethyl-silyl)ethoxycarbonyl (Teoc), 2-(4-trifluoromethylphenylsulfonyl)ethoxycarbonyl (Tsc), tert-butoxycarbonyl (Boc), 1-adamantyloxycarbonyl (Adoc), 2-adamantyl-carbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBoc), vinyl, 2-chloroethyl, 2-phenylsulfonylethyl, p-nitrophenylsulfonyl, p-toluenesulfonyl, phenylsulfonyl, methanesulfonyl, allyl, benzyl, 2-nitrobenzyl, 4-nitrobenzyl, diphenyl-4-pyridylmethyl, N′,N′-dimethylhydrazino, methoxymethyl, tert-butoxymethyl (Bum), benzyloxymethyl (Bom), 2-tetrahydropyranyl (THP), tri(C1-4alkyl)silyl, 1,1-diethoxymethyl, 2-(trimethylsilyl)ethoxymethyl (SEM), N-pivaloyloxymethyl (POM) and the like.

In one embodiment of the present invention provides an improved process for the preparation of ruxolitinib or a salt thereof, comprising:
a) reacting a compound of formula 2:

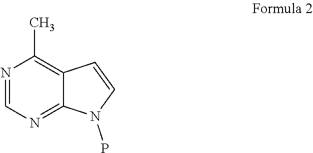

wherein, ‘P’ represents amino protecting group;
with a reagent prepared in-situ from triflic anhydride and DMF in the presence of a suitable solvent to provide a compound of formula 3;

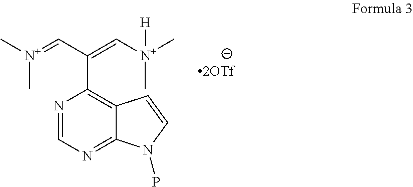

b) contacting the compound of formula 3 with a compound of formula 4 or salt thereof,

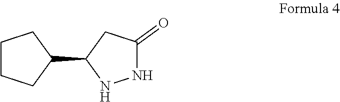 [SMILES: O=C1C[C@H](C2CCCC2)NN1]

to provide a compound of formula 5;

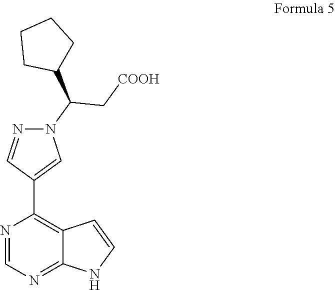 [SMILES: O=C(O)C[C@H](C1CCCC1)n1cc(-c2ncnc3[nH]ccc23)cn1]

c) optionally, purifying the acid intermediate of formula 5 by reacting with dicyclohexylamine (DCHA) in a suitable solvent to provide dicyclohexylamine salt of acid intermediate of formula 5a, and

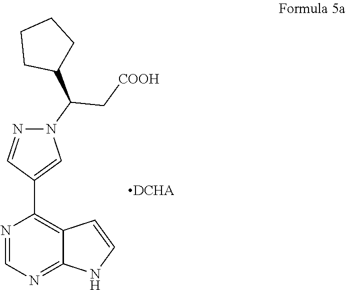 [SMILES: O=C(O)C[C@H](C1CCCC1)n1cc(-c2ncnc3[nH]ccc23)cn1]

further converting into its free base by reacting it with base to provide pure acid intermediate of formula 5;
d) reacting the compound of formula 5 with carbonyldiimidazole (CDI) in a suitable solvent, followed by a reaction with aqueous ammonia to provide a compound of formula 6;

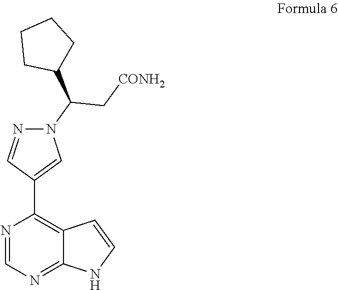 [SMILES: NC(=O)C[C@H](C1CCCC1)n1cc(-c2ncnc3[nH]ccc23)cn1]

e) treating the compound of formula 6 with trifluoroacetic anhydride in the presence of a base and a suitable solvent to provide ruxolitinib; and

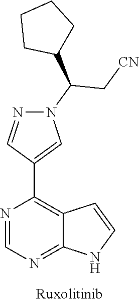 [SMILES: N#CC[C@H](C1CCCC1)n1cc(-c2ncnc3[nH]ccc23)cn1]

f) optionally converting ruxolitinib into its salts thereof.

The starting compounds of formula 2 and formula 4 can be prepared by the methods known in the prior art or by the process described herein the present invention.

The step a) of process involves the reaction of compound of formula 2 (wherein, ‘P’ represents amine protecting group defined above; preferably benzyloxycarbonyl, tert-butoxycarbonyl or N-pivaloyloxymethyl; more preferably benzyloxycarbonyl) with a reagent prepared in-situ from triflic anhydride and DMF in the presence of a suitable solvent under appropriate reaction conditions to provide a compound of formula 3.

The suitable solvent used in the above process is as defined above and preferably selected from nitriles such as acetonitrile, propionitrile and the like; most preferably acetonitrile.

The said reaction is carried out at a suitable temperature of about 0° C. to about 50° C. for sufficient period of time; preferably for about 10 to 16 hours at about 5° C. to about 35° C.

In the present application, the importance of using amino-protecting group have advantageous properties selected from at least one of: chemical purity, introduced high yields without any side reactions, stable under various reaction conditions, improvement in solubility and reduce polarity, and make structural analysis easier.

In the prior known processes, salts of protected compound of formula 3, prepared by using oxalyl chloride and the reaction was carried out at higher temperatures and which in turn salts of compound of formula 3 unstable, and it leads to cleavage of the protecting group and subsequently to form of N-formyl compound as an impurity.

However, the inventors of the present invention surprisingly found that, while using the reagent prepared in-situ from triflic anhydride and DMF in the preparation of compound of formula 3, the reaction can be carried out at lower temperature which helps in avoiding the de-protection and also in eliminating the formation of N-formyl impurity, one of the plausible impurities when amine is not protected with a protecting group.

The step b) of process involves the reaction of the compound of formula 3 with a compound of formula 4 or salt thereof, can be carried out in the presence of a suitable deprotecting agent under appropriate reaction conditions to provide a compound of formula 5.

The deprotecting agent used under an acidic condition is selected from trifluoroacetic acid, trifluoroacetic anhydride, lithium tetrafluoroborate, boron trifluoride-diethyl etherate, aq. HCl and aq. H₂SO₄ and the like; preferably aq. HCl or aq. H₂SO₄.

The deprotecting agent used under a basic condition is selected from aqueous or alcoholic solution of alkali metal hydroxides or alkaline earth metal hydroxide such as sodium hydroxide, potassium hydroxide, lithium hydroxide, magnesium hydroxide, barium hydroxide, calcium hydroxide, beryllium hydroxide, strontium hydroxide, radium hydroxide and the like; preferably aqueous sodium hydroxide or methanolic sodium hydroxide.

The said reaction is carried out at a suitable temperature of about 40° C. to about 90° C. for sufficient period of time; preferably for about 20 to 24 hours at about 50° C. to about 80° C.

The step c) of process involves the purification of acid intermediate of formula 5 by reacting with dicyclohexylamine in a suitable solvent to provide dicyclohexylamine salt of acid intermediate of formula 5a, and then further converting into its free base by reacting it with base under appropriate reaction conditions followed by pH adjustment using aq. acid to provide a pure acid intermediate of formula 5.

In the aforementioned process, the base used is selected from alkali metal hydroxides or alkaline earth metal hydroxide such as sodium hydroxide, potassium hydroxide, lithium hydroxide, magnesium hydroxide, barium hydroxide, calcium hydroxide, beryllium hydroxide, strontium hydroxide, radium hydroxide and the like; preferably sodium hydroxide; and the suitable solvent used is selected from ketone solvents such as acetone, methyl isobutyl ketone, methyl ethyl ketone and the like; preferably acetone.

The said reaction is carried out at a suitable temperature of about 15° C. to about 60° C. for sufficient period of time; preferably for about 1 to 4 hours at about 25° C. to about 55° C.

The step d) of process involves the reaction of the compound of formula 5 with carbonyldiimidazole in a suitable solvent, followed by a reaction with aqueous ammonia under appropriate reaction conditions to provide a compound of formula 6.

The suitable solvent used in the above process is as defined above but also selected from amides such as dimethylformamide, dimethylacetamide and the like; preferably dimethylacetamide or dimethylformamide.

The said reaction is carried out at a suitable temperature of about 0° C. to about 60° C. for sufficient period of time; preferably for about 1 to 4 hours at about 0° C. to about 30° C.

The step e) of process involves the reaction of the compound of formula 6 with trifluoroacetic anhydride in the presence of a base and a suitable solvent under appropriate reaction conditions to provide ruxolitinib.

In another embodiment, the molar ratio of trifluoroacetic anhydride to compound of formula 6 is about 1.1 to 5 molar equivalents; preferably 2 to 4 molar equivalents.

The base and the suitable solvent used in above process is as defined above but also selected from organic bases such as triethylamine, methylamine, ethylamine, 1,8-diazabicycle[5.4.0]undec7-ene, 1,5-diazabicyclo(4.3.0)non-5-ene, lithiumdiisopropyl-amine, n-butyl lithium, tribenzylamine, isopropyl amine, diisopropylamine, diisopropylethyl amine, N-methylmorpholine, N-ethylmorpholine, piperidine, dimethyl amino pyridine, morpholine, pyridine, 2,6-lutidine, 2,4,6-collidine, imidazole, 1-methylimidazole, 1,2,4-triazole, 1,4-diazabicyclo[2.2.2]octane and the like; preferably N-methylmorpholine; and the suitable solvent used is selected from esters such as ethyl acetate, isopropyl acetate and the like or halogenated solvents such as dichloromethane, chloroform, chlorobenzene, bromobenzene and the like; preferably ethyl acetate, isopropyl acetate or dichloromethane.

The said reaction is carried out at a suitable temperature of about 0° C. to about 60° C. for sufficient period of time; preferably for about 1 to 4 hours at about 10° C. to about 30° C.

In the prior known processes, ruxolitinib produced from compound of formula 6, by using phosphorous oxychloride/N-Methyl-2-pyrrolidone (NMP) or oxalyl chloride, and the present inventors found that the reported reaction conditions give low purity of crude ruxolitinib is about 85% and it is around 95% after column chromatography. The major impurities (6-7%) are assumed to be NMP adducts based on LC-MS. Furthermore, chromatography is not suitable for large scale production.

However, the inventors of the present invention surprisingly found that, while using trifluoroacetic anhydride in the preparation of ruxolitinib from compound of formula 6, the reaction conditions are mild, operation friendly and industrially applicable, and could achieve directly high purity of greater than 99% without any further purification steps or column chromatography.

Hence, the inventors of the present invention avoided laborious purification techniques like column chromatography and provided an improved process, which is simple, cost effective and industrially feasible.

In one embodiment, below table illustrates the comparison of prior art process vs present invention with respect to rate of conversion (amide compound of formula 6 into ruxolitinib) and purity of ruxolitinib:

| | | | Rate of | |
|---|---|---|---|---|
| S. No | Reference | Reagent | conversion | Purity |
| 1. | Prior art | POCl₃/NMP | 83% | 85% (crude); |
| | process | | | 95% (after |
| | | | | purification) |
| 2. | Present | TFAA/NMM | 97% | 99.27% (crude); |
| | invention | | | Above 99.65% (after |
| | | | | purification) |

The step f) of process involves the conversion of ruxolitinib into its salts thereof can be carried out by treating ruxolitinib with an acid component like phosphoric acid in a suitable solvent under appropriate reaction conditions to provide ruxolitinib phosphate.

The suitable solvent used in the above process is as defined above but also selected from alcohols such as methanol, ethanol, isopropanol, n-butanol, iso-butanol and the like; preferably isopropanol.

The said reaction is carried out at a suitable temperature of about 20° C. to about 60° C. for sufficient period of time; preferably for about 1 to 2 hours at about 20° C. to about 30° C.

The intermediate compound of formula 3, formula 5 or formula 5a obtained in the present invention can be optionally isolated.

In another embodiment of the present invention provides improved process for the preparation of intermediate compound of formula 3, comprising:
reacting a compound of formula 2:

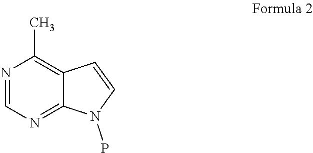

wherein, ‘P’ represents amine protecting group;
with a reagent prepared in-situ from triflic anhydride and DMF in the presence of a suitable solvent to provide a compound of formula 3.

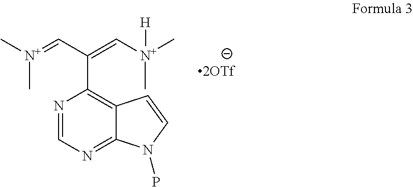

In the present invention, the suitable solvent used in the above process is as defined above but also selected from nitriles such as acetonitrile, propionitrile and the like; preferably acetonitrile.

The said reaction is carried out at a suitable temperature of about 0° C. to about 50° C. for sufficient period of time; preferably for about 10 to 16 hours at about 5° C. to about 35° C.

In another embodiment, the intermediate compound of formula 3 obtained according to the above embodiment is further converted to ruxolitinib or a salt thereof.

In another embodiment of the present invention provides an improved process for the preparation of ruxolitinib or a salt thereof, comprising:
a) reacting a compound of formula 5:

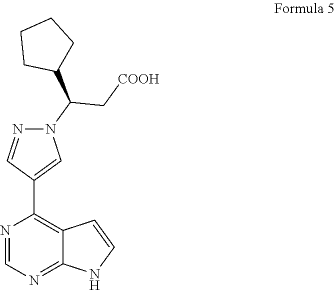 [SMILES: O=C(O)C[C@H](C1CCCC1)n1cc(-c2ncnc3[nH]ccc23)cn1]

with carbonyldiimidazole (CDI) in a suitable solvent, followed by a reaction with aqueous ammonia to provide a compound of formula 6;

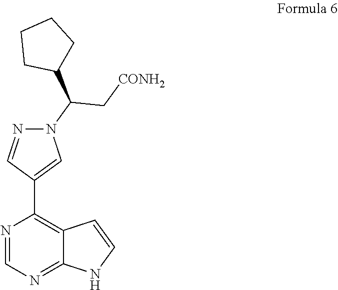 [SMILES: NC(=O)C[C@H](C1CCCC1)n1cc(-c2ncnc3[nH]ccc23)cn1]

b) reacting the compound of formula 6 with trifluoroacetic anhydride in the presence of a base and a suitable solvent to provide ruxolitinib; and

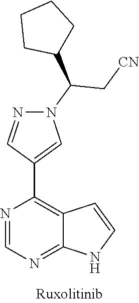 [SMILES: N#CC[C@H](C1CCCC1)n1cc(-c2ncnc3[nH]ccc23)cn1]

c) optionally converting ruxolitinib into its salts thereof.

Step a) of process involves the reaction of the compound of formula 5 with carbonyldiimidazole in a suitable solvent, followed by a reaction with aqueous ammonia under appropriate reaction conditions to provide a compound of formula 6. The suitable solvent used in the above process is as defined above but also selected from amides such as dimethylformamide, dimethylacetamide and the like; preferably dimethylacetamide or dimethylformamide.

The said reaction is carried out at a suitable temperature of about 0° C. to about 60° C. for sufficient period of time; preferably for about 1 to 4 hours at about 0° C. to about 30° C.

Step b) of process involves the reaction of the compound of formula 6 with trifluoroacetic anhydride in the presence of a base and a suitable solvent under appropriate reaction conditions to provide ruxolitinib.

In another embodiment, the molar ratio of trifluoroacetic anhydride to compound of formula 6 is about 1.1 to 5 molar equivalents; preferably 2 to 4 molar equivalents.

In addition to the base and the suitable solvent used in the process described above, the base can also be selected from organic bases such as triethylamine, methylamine, ethylamine, 1,8-diazabicycle[5.4.0]undec7-ene, 1,5-diazabicyclo(4.3.0)non-5-ene, lithiumdiisopropyl-amine, n-butyl lithium, tribenzylamine, isopropyl amine, diisopropylamine, diisopropylethyl amine, N-methylmorpholine, N-ethylmorpholine, piperidine, dimethyl amino pyridine, morpholine, pyridine, 2,6-lutidine, 2,4,6-collidine, imidazole, 1-methylimidazole, 1,2,4-triazole, 1,4-diazabicyclo[2.2.2]octane and the like; preferably N-methylmorpholine; and the suitable solvent used is selected from esters such as ethyl acetate, isopropyl acetate and the like or halogenated solvents such as dichloromethane, chloroform, chlorobenzene, bromobenzene and the like; preferably ethyl acetate, isopropyl acetate or dichloromethane.

The said reaction is carried out at a suitable temperature of about 0° C. to about 60° C. for sufficient period of time; preferably for about 1 to 4 hours at about 10° C. to about 30° C.

Step c) of process involves the conversion of ruxolitinib into its salts thereof can be carried out by treating ruxolitinib with an acid component like phosphoric acid in a suitable solvent under appropriate reaction conditions to provide ruxolitinib phosphate.

The suitable solvent used in the above process is as defined above but also selected from alcohols such as methanol, ethanol, isopropanol, n-butanol, iso-butanol and the like; preferably isopropanol.

The said reaction is carried out at a suitable temperature of about 20° C. to about 60° C. for sufficient period of time; preferably for about 1 to 2 hours at about 20° C. to about 30° C.

In another embodiment of the present invention provides improved process for the preparation of ruxolitinib or a salt thereof, comprising:
a) reacting a compound of formula 6:

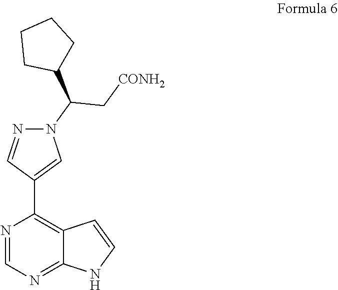 [SMILES: NC(=O)C[C@H](C1CCCC1)n1cc(-c2ncnc3[nH]ccc23)cn1]

with trifluoroacetic anhydride in the presence of a base and a suitable solvent to provide ruxolitinib; and

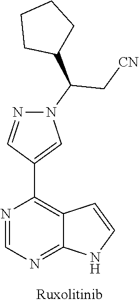 [SMILES: N#CC[C@H](C1CCCC1)n1cc(-c2ncnc3[nH]ccc23)cn1]

b) optionally converting ruxolitinib into its salts thereof.

Step a) of process involves the reaction of the compound of formula 6 with trifluoroacetic anhydride in the presence of a base and a suitable solvent under appropriate reaction conditions to provide ruxolitinib.

In another embodiment, the molar ratio of trifluoroacetic anhydride to compound of formula 6 is about 1.1 to 5 molar equivalents; preferably 2 to 4 molar equivalents.

In the present invention, the base and the suitable solvent used in above step a) process is as defined above but also selected from organic bases such as triethylamine, methylamine, ethylamine, 1,8-diazabicycle[5.4.0]undec7-ene, 1,5-diazabicyclo(4.3.0)non-5-ene, lithiumdiisopropylamine, n-butyllithium, tribenzylamine, isopropyl amine, diisopropylamine, diisopropylethyl amine, N-methylmorpholine, N-ethylmorpholine, piperidine, dimethyl amino pyridine, morpholine, pyridine, 2,6-lutidine, 2,4,6-collidine, imidazole, 1-methylimidazole, 1,2,4-triazole, 1,4-diazabicyclo[2.2.2]octane and the like; preferably N-methylmorpholine; and the suitable solvent used is selected from esters such as ethyl acetate, isopropyl acetate and the like or halogenated solvents such as dichloromethane, chloroform, chlorobenzene, bromobenzene and the like; preferably ethyl acetate, isopropyl acetate or dichloromethane.

The said reaction is carried out at a suitable temperature of about 0° C. to about 60° C. for sufficient period of time; preferably for about 1 to 4 hours at about 10° C. to about 30° C.

Step b) of process involves the conversion of ruxolitinib into its salts thereof can be carried out by treating ruxolitinib with an acid component like phosphoric acid in a suitable solvent under appropriate reaction conditions to provide ruxolitinib phosphate.

In the present invention, the suitable solvent used in above step b) process is as defined above but also selected from alcohols such as methanol, ethanol, isopropanol, n-butanol, iso-butanol and the like; preferably isopropanol.

The said reaction is carried out at a suitable temperature of about 20° C. to about 60° C. for sufficient period of time; preferably for about 1 to 2 hours at about 20° C. to about 30° C.

### Crystalline Form G of Ruxolitinib

In another embodiment of the present invention provides a novel crystalline Form G of ruxolitinib.

In another embodiment of the present invention provides a novel crystalline Form G of ruxolitinib is characterized by its PXRD pattern having one or more peaks at about 10.1, 13.6, 17.5, and 22.8±0.2° 2θ.

In another embodiment of the present invention provides a novel crystalline Form G of ruxolitinib is further characterized by its PXRD pattern having one or more peaks at about 6.5, 9.3, 10.1, 11.1, 12.0, 13.6, 14.41, 17.5, 22.8±0.2° 2θ.

The novel crystalline Form G of ruxolitinib exhibits the following diffraction peaks in PXRD pattern, see Table 1, below:

TABLE 1

| Position [° 2θ] | d-spacing [Å] | Relative Intensity [%] |
|---|---|---|
| 6.5 | 13.57 | 0.90 |
| 9.3 | 9.407 | 7.31 |
| 10.1 | 8.676 | 5.55 |
| 11.1 | 7.9434 | 19.52 |
| 12.0 | 7.315 | 0.48 |
| 13.0 | 6.7822 | 2.81 |
| 13.6 | 6.4778 | 24.47 |
| 13.9 | 6.3438 | 24.57 |
| 14.4 | 6.1392 | 18.16 |
| 14.6 | 6.044 | 10.80 |
| 16.5 | 5.3641 | 23.31 |
| 17.5 | 5.0529 | 35.29 |
| 18.0 | 4.9083 | 15.72 |
| 18.7 | 4.7324 | 17.90 |
| 19.3 | 4.5893 | 41.58 |
| 20.3 | 4.3596 | 16.53 |
| 20.7 | 4.2682 | 6.15 |
| 21.5 | 4.1224 | 100 |
| 22.5 | 3.932 | 54.25 |
| 22.8 | 3.889 | 35.61 |
| 24.3 | 3.653 | 5.78 |
| 25.0 | 3.557 | 32.57 |
| 25.3 | 3.5048 | 32.24 |
| 25.8 | 3.4374 | 6.69 |
| 26.2 | 3.3955 | 5.40 |
| 26.6 | 3.348 | 6.61 |
| 28.3 | 3.145 | 6.66 |
| 29.1 | 3.0588 | 1.28 |
| 29.6 | 3.0115 | 2.20 |
| 30.2 | 2.9475 | 2.50 |

In another embodiment of the present invention provides a novel crystalline Form G of ruxolitinib is characterized by a DSC thermogram having exothermic peak at about 138-146° C.

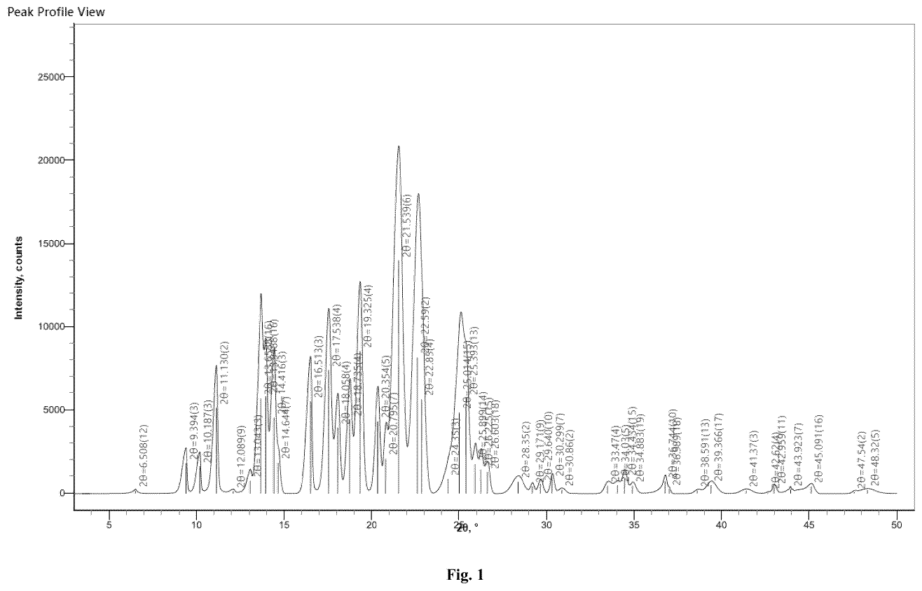

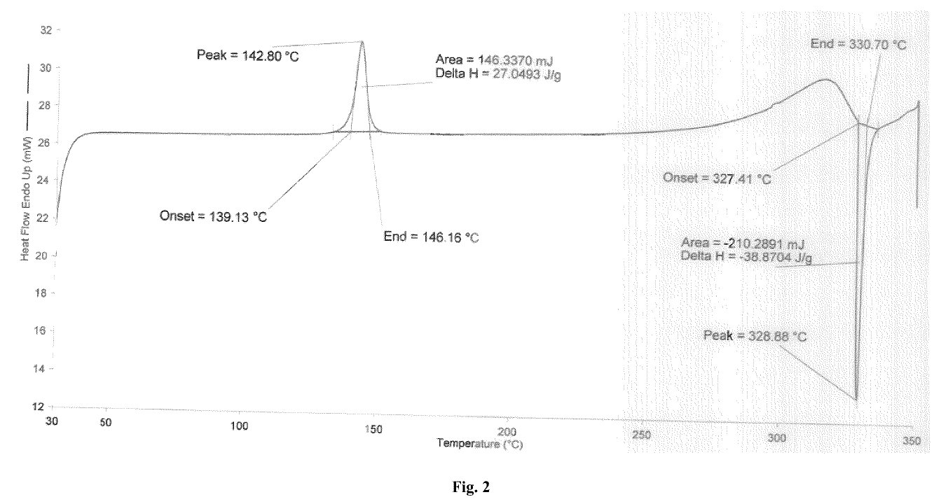

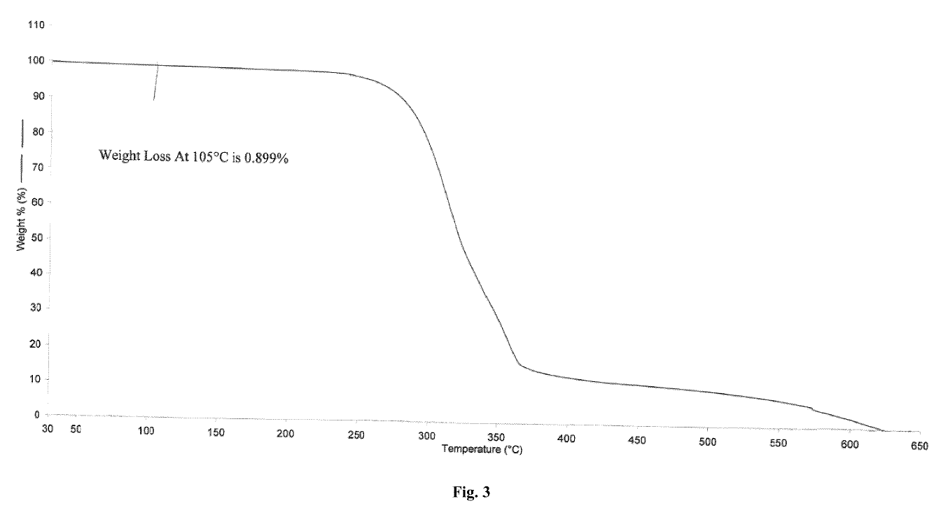

The crystalline form G of ruxolitinib is further characterized by an XRPD pattern, a DSC thermogram, and a TGA as depicted in FIG. 1, FIG. 2, and FIG. 3 respectively.

The novel polymorphic form of ruxolitinib form G of the present invention has advantageous properties selected from at least one of: chemical purity, flowability, solubility, morphology or crystal habit, stability-such as storage stability.

In another embodiment of the present invention provides a process for the preparation of novel crystalline Form G of ruxolitinib, comprising:
a) dissolving ruxolitinib in a suitable solvent at a suitable temperature; and
b) isolating the crystalline Form G of ruxolitinib.

In step a) of the aforementioned process, dissolving ruxolitinib in a suitable solvent at a suitable temperature from about 15° C. to boiling point of the solvent used; preferably at about 30° C. to about 160° C.; wherein the suitable solvent is as defined above but also selected from aprotic solvents of the group comprising ethers, amides, esters, nitriles, sulfoxides, ketones, halogenated solvents and the like; preferably esters such as ethyl acetate, isopropyl acetate and the like or halogenated solvents such as dichloromethane, chloroform, chlorobenzene, bromobenzene and the like; more preferably ethyl acetate, isopropyl acetate or dichloromethane; and isolation of crystalline Form G of ruxolitinib in step b) can be carried out by any conventional techniques known in the art, for example filtration, evaporation or decantation of solution then followed by drying.

In another embodiment of the present invention provides a process for the preparation of novel crystalline Form G of ruxolitinib, comprising:
a) dissolving ruxolitinib in a suitable solvent at a suitable temperature;
b) optionally treating with activated carbon;
c) adding a suitable anti-solvent to step b) solution at a suitable temperature; and
d) isolating the crystalline Form G of ruxolitinib.

In step a) of the aforementioned process, dissolving ruxolitinib in a suitable solvent at a suitable temperature from about 15° C. to reflux temperature of the solvent used; preferably at about 30° C. to about 160° C.; wherein the suitable solvent is as defined above but also selected from aprotic solvents of the group comprising ethers, amides, esters, nitriles, sulfoxides, ketones, halogenated hydrocarbons and the like; preferably esters such as ethyl acetate, isopropyl acetate and the like or halogenated solvents such as dichloromethane, chloroform, chlorobenzene, bromobenzene and the like; more preferably ethyl acetate, isopropyl acetate or dichloromethane; and then optionally treated with activated charcoal in step b). In step c) the suitable anti-solvent was added to step b) solution at a suitable temperature from about 20° C. to about 60° C. for a sufficient period of time up to complete crystallization; preferably at about 25° C. to 55° C.; wherein the suitable anti-solvent is selected from but not limited to hydrocarbon solvents such as heptane, hexane, cyclohexane, cycloheptane, toluene, xylene, cyclohexane and the like or ethers such as methyl tert-butyl ether, diisopropyl ether, diethyl ether tetrahydrofuran, 2-methyl tetrahydrofuran, cyclopentyl methyl ether, dioxane and the like; preferably hexane, heptane or methyl tert-butyl ether; and finally in step d) the isolation of crystalline Form G of ruxolitinib can be carried out by any conventional techniques known in the art, for example filtration, evaporation or decantation of solution then followed by drying.

In another embodiment of the present invention provides a novel crystalline Form G of ruxolitinib, which is prepared by a process involves single solvent crystallization technique and/or solvent and anti-solvent crystallization technique.

In another embodiment of the present invention provides a process for the preparation of a crystalline form G of ruxolitinib comprising reacting ruxolitinib acid addition salt with a base in a suitable solvent under appropriate reaction conditions to provide a crystalline form G of ruxolitinib.

In the aforementioned process, the ruxolitinib acid addition salt is selected from phosphoric acid, maleic acid, sulfuric acid, benzene sulfonic acid, oxalic acid, hydrobromic acid, hydrochloric acid, citric acid, fumaric acid, tartaric acid, p-toluenesulphonic acid, benzoic acid, benzenesulphonic acid, ethanesulphonic acid, 2-naphthalenesulphonic acid, 4-chlorobenzenesulphonic acid and the like; preferably phosphoric acid or hydrochloric acid.

In some embodiments, the base used is selected from alkali metal carbonates such as sodium carbonate, potassium carbonate, lithium carbonate and the like; preferably potassium carbonate; and the suitable solvent is selected from esters such as ethyl acetate, isopropyl acetate and the like or halogenated solvents such as dichloromethane, chloroform, chlorobenzene, bromobenzene and the like; preferably ethyl acetate, isopropyl acetate or dichloromethane.

In some embodiments, the base used from about 1 to about 10 molar equivalents of the base relative to ruxolitinib acid addition salt. In some embodiments, the base used from about 1 to about 5 molar equivalents of the base relative to ruxolitinib acid addition salt. In some embodiments, the base used from about 1 to about 3 molar equivalents of the base relative to ruxolitinib acid addition salt.

In some embodiments, the reacting of ruxolitinib acid addition salt with a base comprises using an amount of the base sufficient to attain a pH of greater than 7.

In other embodiment ruxolitinib acid addition salt is dissolved in a suitable solvent at a suitable temperature from about 15° C. to reflux temperature of the solvent used; in some embodiments the isolation of crystalline form G of ruxolitinib can be carried out by any conventional technique known in the art, for example filtration, evaporation or decantation of solution then followed by drying.

In another embodiment of the present invention provides a process for the preparation of a crystalline form G of ruxolitinib comprising reacting ruxolitinib phosphate salt with a base in a solvent component.

In some embodiments, the base used is selected from alkali metal carbonates such as sodium carbonate, potassium carbonate, lithium carbonate and the like; preferably potassium carbonate; and the suitable solvent is selected from esters such as ethyl acetate, isopropyl acetate and the like or halogenated solvents such as dichloromethane, chloroform, chlorobenzene, bromobenzene and the like; preferably ethyl acetate, isopropyl acetate or dichloromethane.

In some embodiments, the base used from about 1 to about 10 molar equivalents of the base relative to ruxolitinib phosphate salt. In some embodiments, the base used from about 1 to about 5 molar equivalents of the base relative to ruxolitinib phosphate salt. In some embodiments, the base used from about 1 to about 3 molar equivalents of the base relative to ruxolitinib phosphate salt.

In some embodiments, the reacting of ruxolitinib phosphate salt with a base comprises using an amount of the base sufficient to attain a pH of greater than 7.

In other embodiment dissolving ruxolitinib phosphate salt in a suitable solvent at a suitable temperature from about 15° C. to boiling point of the solvent used; in some embodiments the isolation of crystalline form G of ruxolitinib can be carried out by any conventional techniques known in the art, for example filtration, evaporation or decantation of solution then followed by drying.

In another embodiment of the present invention provides a process for the preparation of ruxolitinib acid addition salt, comprising: reacting crystalline ruxolitinib Form G with an acid component in a suitable solvent under appropriate reaction conditions to provide ruxolitinib acid addition salt.

In the aforementioned process, reacting crystalline ruxolitinib Form G with an acid component is selected from phosphoric acid, maleic acid, sulfuric acid, benzene sulfonic acid, oxalic acid, hydrobromic acid, hydrochloric acid, citric acid, fumaric acid, tartaric acid, p-toluenesulphonic acid, benzoic acid, benzenesulphonic acid, ethanesulphonic acid, 2-naphthalenesulphonic acid, 4-chlorobenzenesulphonic acid and the like; preferably phosphoric acid or hydrochloric acid; in a suitable solvent under appropriate reaction conditions to provide ruxolitinib acid addition salt.

In the present invention, the suitable solvent used in the above process is as defined above but also selected from alcohols, esters, nitriles, sulfoxides, ketones, hydrocarbons and halogenated solvents; preferably alcohols such as methanol, ethanol, iso-propanol, n-butanol, iso-butanol and the like or esters such as ethyl acetate, isopropyl acetate and the like; more preferably methanol, ethanol, iso-propanol, ethyl acetate or isopropyl acetate.

The said reaction is carried out at a suitable temperature of about 0° C. to about 60° C. for sufficient period of time; preferably for about 1 to 4 hours at about 25° C. to about 55° C.

The crystalline form G of ruxolitinib of the present invention may be administered as part of a pharmaceutical composition for the treatment of a JAK-associated disease or disorder.

Accordingly, in a further aspect, there is provided a pharmaceutical composition comprising the crystalline form G of ruxolitinib of the present invention, one or more pharmaceutically acceptable carriers, diluents, or excipients, and optionally other therapeutic ingredients.

Pharmaceutical compositions comprising the crystalline form G of ruxolitinib of the present invention may be administered orally, topically, parenterally, by inhalation or spray, rectally, or in the form of injectables.

### Process for the Preparation of Crystal Modification 3 of Ruxolitinib Hydrochloride Salt

Another embodiment of the present invention provides an improved process for the preparation of crystal modification 3 of ruxolitinib hydrochloride salt, comprising:

reacting ruxolitinib with hydrochloric acid in a mixture of ethyl acetate and ethanol solvent system under appropriate reaction conditions to provide crystalline modification 3 of ruxolitinib hydrochloride salt.

The term “crystal modification” of ruxolitinib, as used herein, is synonymous to commonly used expressions “polymorphic form” or “crystalline form” of ruxolitinib.

As stated under the background section, WO2017008772A1 describes preparation of crystal modification 3 of ruxolitinib hydrochloride salt. But the prior art processes do not produce crystal modification 3 of ruxolitinib hydrochloride salt in a single step reaction i.e., reaction of ruxolitinib with hydrochloric acid step. They involve multiple steps, including first isolating crystal modification 1, converting crystal modification 1 to crystal modification 2, and then converting crystal modification 2 to crystal modification 3. It is evident that these prior art processes are very laborious and commercially not feasible.

The present inventors have surprisingly developed a simple, single step process to produce crystal modification 3 of ruxolitinib hydrochloride by using specific solvent mixture and volume ratios of the solvent system.

Accordingly, in one embodiment, the volume ratio of ethanol and ethyl acetate is about 1:1 to 1:10.

In another embodiment, reaction of ruxolitinib with hydrochloric acid is carried out at a suitable temperature of about 0° C. to about 50° C. for sufficient period of time; preferably for about 2 to 6 hours at about 20° C. to about 40° C.

In another embodiment, isolation of crystal modification 3 of ruxolitinib hydrochloride by conventional methods known in the art for example by cooling or addition of anti-solvent.

In another embodiment, isolation of crystal modification 3 of ruxolitinib hydrochloride by addition of anti-solvent like ethyl acetate.

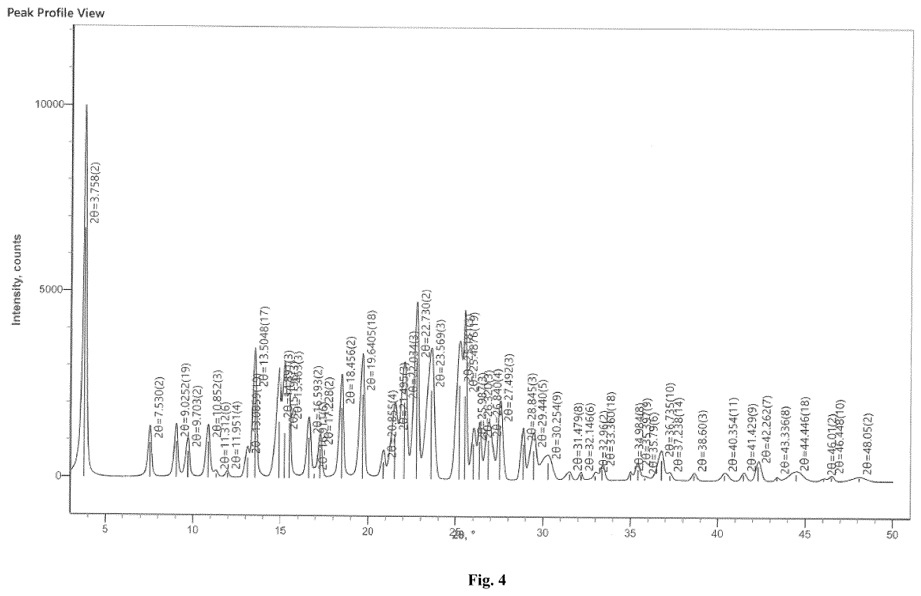

FIG. 4 illustrates X-Ray Powder Diffraction (XRPD) pattern of the crystal modification 3 of ruxolitinib hydrochloride.

In the foregoing section, embodiments are described by way of examples to illustrate the processes of invention. However, these are not intended in any way to limit the scope of the present invention. Variants of the examples that would be evident to a person ordinarily skilled in the art are within the scope of the present invention.

### EXAMPLES

The process details of the invention are provided in the examples given below, which are provided by way of illustration only and therefore should not be construed to limit the scope of the invention.

### Example-1: Preparation of (R)-3-(4-(7H-Pyrrolo[2,3-d]pyrimidin-4-yl)-1H-pyrazol-1-yl)-3-cyclopentylpropanoic acid dicyclohexylamine

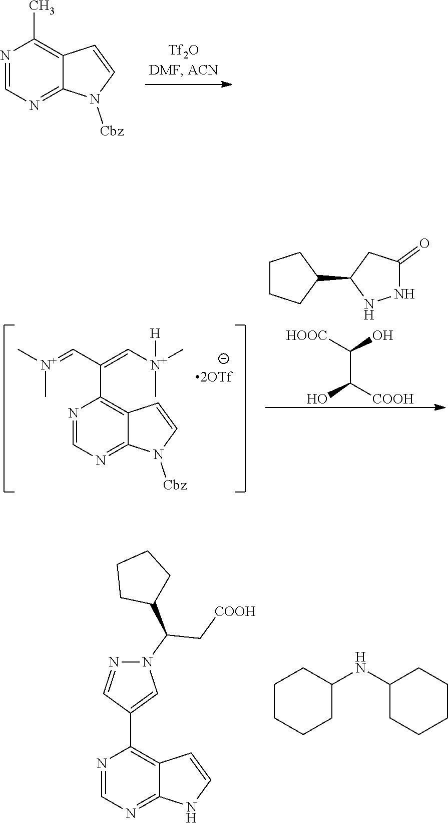

Acetonitrile (150 ml) and DMF (184.5 ml) were charged into round bottom flask (RBF) at Room Temperature (RT). To the reaction mass trifluromethanesulphonic anhydride (237.5 ml) was added and maintained the reaction mass for 2 to 3 hours at RT. Benzyl 4-methyl-7H-pyrrolo[2,3-d]pyrimidine-7-carboxylate (50 g) was charged into the reaction mass at below 10° C. and maintained the reaction mass for until completion at below 20° C. Water (100 ml) was added to the reaction mass at below 20° C. and adjusted the reaction mass pH to 2.5-3.5 with aq. NaOH solution (10 V). (R)-5-cyclopentylpyrazolidin-3-one D-tartrate (68.32 g) was charged into the reaction mass at below 20° C., stirred the reaction mass at 50-70° C. until completion

The reaction mass was cooled to below 30° C., adjusted the reaction mass pH to 3.0-5.0 with aq. NaOH solution extracted the compound with DCM and washed the DCM layer with brine solution. Distilled off the solvent completely under vacuum below 40° C. and charged aq. HCl, stirred the reaction mass at 50 to 70° C. until completion of the reaction. Adjusted the reaction mass pH to 10.0-12.0 with aq. Sodium hydroxide solution at RT and stirred the reaction mass at 40-60° C. until completion of the reaction. Cooled the reaction mass to RT, washed the reaction mass with ethyl acetate, and extracted the compound with Methanolic Ethyl acetate at pH to 3.5-6.0. The organic layer was washed with aq. NaCl solution and distilled off the solvent under reduced pressure to give crude compound. The crude compound was dissolved in acetone and treated with charcoal and DCHA (33.91 g) was added at 40 to 50° C. The resulting mass was maintained at reflux, cooled to RT and filtered and purified the material using acetone to get DCHA salt of (R)-3-(4-(7H-Pyrrolo[2,3-d]pyrimidin-4-yl)-1H-pyrazol-1-yl)-3-cyclopentylpropanoic acid. Yield: 40-45 g

### Example-2: Preparation of (R)-3-(4-(7H-Pyrrolo[2,3-d]pyrimidin-4-yl)-1H-pyrazol-1-yl)-3-cyclopentyl propionamide

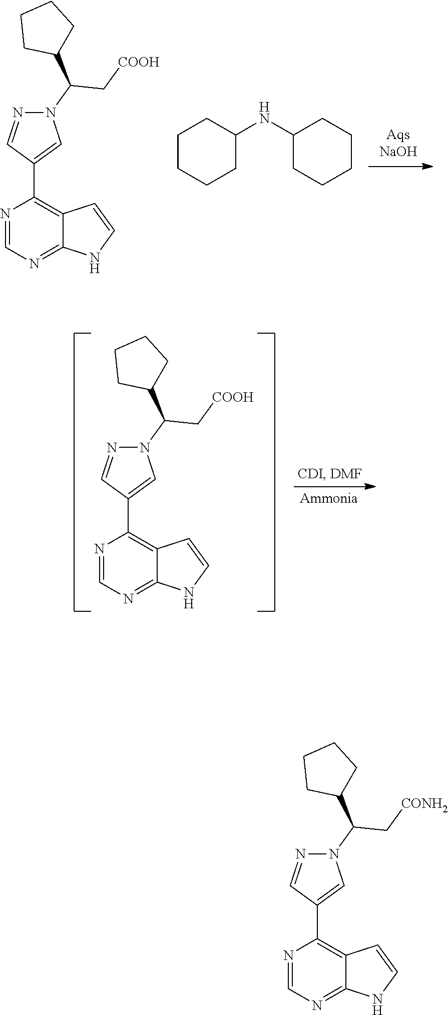

DCHA salt of (R)-3-(4-(7H-Pyrrolo[2,3-d]pyrimidin-4-yl)-1H-pyrazol-1-yl)-3-cyclopentylpropanoic acid (100 g) was taken into water and pH was adjusted to >11 using Aq. NaOH. The mass was washed and adjusted the pH 3.5 to 5.5 and extracted with acetonitrile ethyl acetate mixture. The combined organic layer was washed with brine, distilled off the solvent. The crude material was taken into DMF (2.5 V). CDI (1.0 eq-4.0 eq) was added into the mass under nitrogen atmosphere at below 15° C. and stirred at the same temperature. After completion of the reaction, ammonia solution was added at below 15° C. and stirred for 1 to 2 hours. Aq. sodium chloride solution was added into reaction mass and stirred the mass and filtered. The material was purified using aq. IPA to obtain (R)-3-(4-(7H-Pyrrolo[2,3-d]pyrimidin-4-yl)-1H-pyrazol-1-yl)-3-cyclopentyl propionamide. Yield: 25-45 g

### Example-3: Preparation and Purification of Ruxolitinib

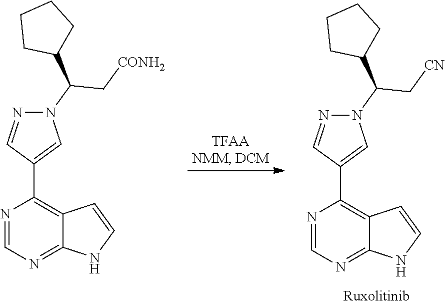 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and DCM (5 V) were charged into RBF at RT under nitrogen atmosphere. 4-Methylmorpholine (4.0 eq) was charged and stirred for 10 to 15 min.

The reaction mass was cooled to below 20° C., trifluoroacetic anhydride (2-3 m eq) was added and stirred until completion of the reaction. After completion of the reaction, the reaction mass was washed with NaHCO₃ solution, water and brine. The organic layer was distilled off under vacuum and isolated from ethyl acetate/n-heptane to get Ruxolitinib (crystalline form G). Yield: 12-18 g

### Example-4: Preparation of Ruxolitinib Phosphate

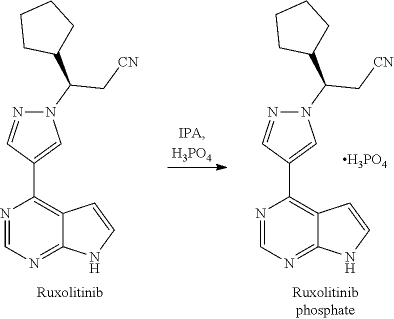

Ruxolitinib (100 g) obtained above, and IPA (30 V) were charged into RBF at 20 to 30° C. The mass was heated to 50 to 60° C. and maintained for 30 to 50 min at the same temperature. The reaction mass was filtered on hyflo bed and washed with IPA. Ortho-phosphoric acid solution (1.0 eq.) was added into reaction mass at 50 to 60° C. and stirred the mass at 50 to 60° C. for 1 to 2 hours. The mass was cooled to 25 to 30° C. and stirred for 1 to 2 hours at 25 to 30° C. The solid obtained was filtered, washed with IPA and dried the wet compound under vacuum at 50 to 60° C. to get Ruxolitinib Phosphate.

Yield: 95 g

### Example-5: Preparation of Ruxolitinib Hydrochloride

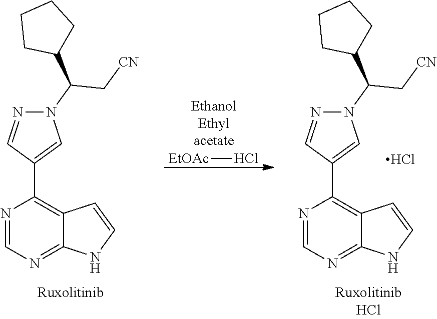

Ruxolitinib (20 g) and ethyl acetate (20 V) were charged into RBF and stirred the mass at 20 to 40° C. for 20 to 30 min. The mass was heated to 40 to 60° C. and stirred for 10 to 40 min. Ethanol (2 V) was charged into the mass at below 50° C., HCl in ethyl acetate (1.0-2.0 V) was added into reaction mass at below 50° C. The mass was stirred until the formation heterogeneous mass. Ethyl acetate (40 V) was added into the reaction mass and stirred for 2 to 4 hours at below 40° C. Cooled the reaction mass temperature to below 10° C. and stirred for 2 to 4 hours. The solid obtained was filtered, washed with ethyl acetate and dried the wet compound under vacuum to get Ruxolitinib hydrochloride crystal modification 3. Yield: 13-16 g

### Example-6: Preparation of Ruxolitinib Hydrochloride

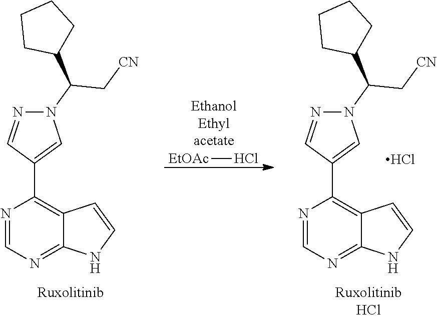

Ruxolitinib (20 g) and ethyl acetate (20 V) were charged into RBF and stirred the mass at 20 to 40° C. for 20 to 30 min. The mass was heated to 40 to 60° C. and stirred for 10 to 40 min. Ethanol (3 V) was charged into the mass at below 50° C., HCl in ethyl acetate (2.0-3.0 V) was added into reaction mass at below 50° C. The mass was stirred until the formation heterogeneous mass. Ethyl acetate (20 V) was added into the reaction mass and stirred for 2 to 4 hours at below 40° C. Cooled the reaction mass temperature to below 10° C. and stirred for 2 to 4 hours. The solid obtained was filtered, washed with ethyl acetate and dried the wet compound under vacuum to get Ruxolitinib hydrochloride crystal modification 3. Yield: 18-20 g

### Example-7: Preparation of Ruxolitinib Hydrochloride

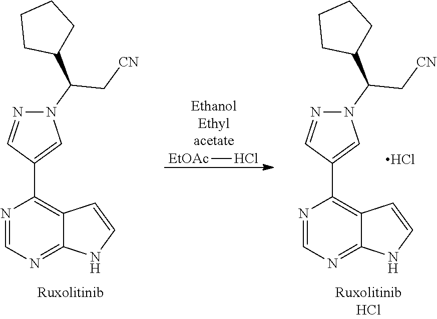

Ruxolitinib (20 g) and ethyl acetate (20 V) were charged into RBF and stirred the mass at 20 to 40° C. for 20 to 30 min. The mass was heated to 40 to 60° C. and stirred for 10 to 40 min. Ethanol (3-4V) was charged into the mass at below 50° C., HCl in ethyl acetate (2.0-3.0 V) was added into reaction mass at below 50° C. The mass was stirred until the formation heterogeneous mass. Ethyl acetate (35-45 V) was added into the reaction mass and stirred for 2 to 4 hours at below 40° C. Cooled the reaction mass temperature to below 10° C. and stirred for 2 to 4 hours. The solid obtained was filtered, washed with ethyl acetate and dried the wet compound under vacuum to get Ruxolitinib hydrochloride crystal modification 3. Yield: 16-18 g

### Example-8: Preparation and Purification of Ruxolitinib

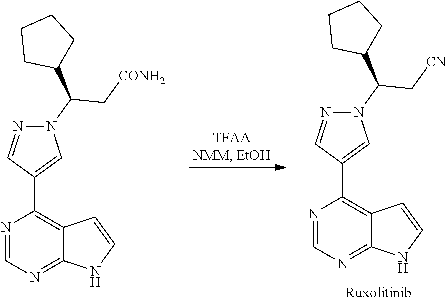 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and ethyl acetate (8 V) were charged into RBF at RT under nitrogen atmosphere, then 4-Methylmorpholine (NMM, 5.0 eq.,) was charged into the reaction mass and stirred for 10-15 min. The reaction mass was cooled to 10-15° C. and trifluoroacetic anhydride (TFAA, 2-3 m eq.,) was added slowly under nitrogen atmosphere at below 20° C. The reaction mass was stirred for 1-2 hours at 15-20° C. under nitrogen atmosphere. The reaction mass was quenched with aq. NaHCO₃ solution (10 V) and stirred for 15-20 min. The layers were separated, and the aqueous layer was extracted with ethyl acetate (10 V). The combined each organic layer was washed with aqueous NaHCO₃ solution (10 V), DM-water (2× 10 V) and aqueous saturated NaCl (6 V) solution at RT. The organic layer was concentrated under vacuum at 50° C. to get Ruxolitinib (crude). Yield: ˜19 g (crude)

The crude compound of Ruxolitinib (19 g) and ethyl acetate (3 V) were charged into RBF at RT. The reaction mass was heated to 45-50° C. and stirred for 30-45 min at same temperature. Then the reaction mass was cooled to 0-5° C. and stirred for 1-2 hours at the same temperature. The solid obtained was filtered and washed with pre-cooled ethyl acetate (1V). The obtained wet material was dried under vacuum to get Ruxolitinib crystalline form G. Yield: 13 to 16 g; Purity: >99%

### Example-9: Preparation and Purification of Ruxolitinib

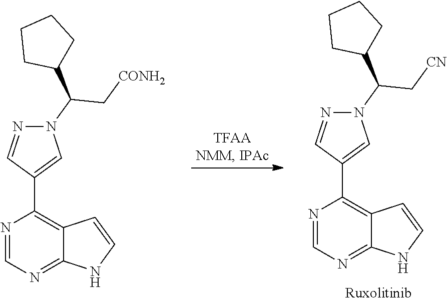 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and isopropyl acetate (8 V) were charged into RBF at RT under nitrogen atmosphere, then 4-Methylmorpholine (NMM, 5.0 eq.,) was charged into the reaction mass and stirred for 10-15 min. The reaction mass was cooled to 10-15° C. and trifluoroacetic anhydride (TFAA, 2-3 m. eq.,) was added slowly under nitrogen atmosphere at below 20° C. The reaction mass was stirred for 1-2 hours at 15-20° C. under nitrogen atmosphere. The reaction mass was quenched with aq. NaHCO₃ solution (10 V) and stirred for 15-20 min. The layers were separated, and the aqueous layer was extracted with isopropyl acetate (10 V). The combined each organic layer was washed with aqueous NaHCO₃ solution (10 V), DM-water (2× 10 V) and aqueous saturated NaCl (6 V) solution at RT. The organic layer was treated with charcoal, filtered and the solvent from the filtrate was distilled-off under reduced pressure at below 50° C. until 4.0±0.5 V remains in the RBF. n-Heptane (3.0 V) was added to the reaction mass at 50±5° C. then cooled to 25-30° C. The mass was stirred for 1-2 hours at 25-30° C., filtered and washed with a mixture of isopropyl acetate: n-Heptane (2V, 1:1 ratio). The obtained wet material was dried under vacuum to get Ruxolitinib crystalline form G. Yield: 13 to 16 g; Purity: >99%

### Example-10: Preparation and Purification of Ruxolitinib

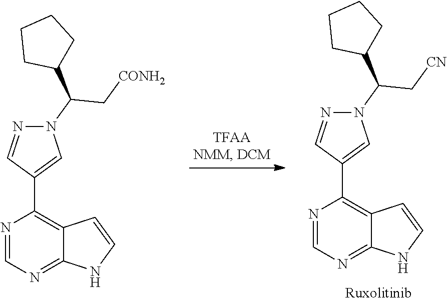 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and dichloromethane (8 V) were charged into RBF at RT under nitrogen atmosphere, then 4-Methylmorpholine (NMM, 5.0 eq.,) was charged into the reaction mass and stirred for 10-15 min. The reaction mass was cooled to 10-15° C. and trifluoroacetic anhydride (TFAA, 2-3 m. eq.,) was added slowly under nitrogen atmosphere at below 20° C. The reaction mass was stirred for 1-2 hours at 15-20° C. under nitrogen atmosphere. The reaction mass was quenched with aq. NaHCO₃ solution (10 V) and stirred for 15-20 min. The layers were separated, and the aqueous layer was extracted with dichloromethane (10 V). The combined each organic layer was washed with aqueous NaHCO₃ solution (10 V), DM-water (2× 10 V) and aqueous saturated NaCl (6 V) solution at RT. The organic layer was treated with charcoal, filtered and the solvent from the filtrate was distilled-off under reduced pressure at below 50° C. until 4.0±0.5 V remains in the RBF. n-Heptane (3.0 V) was added to the reaction mass at 50±5° C. then cooled to 25-30° C. The mass was stirred for 1-2 hours at 25-30° C., filtered and washed with a mixture of dichloromethane: n-Heptane (2V, 1:1 ratio). The obtained wet material was dried under vacuum to get Ruxolitinib crystalline form G. Yield: 13 to 16 g; Purity: >99%

### Example-11: Preparation and Purification of Ruxolitinib

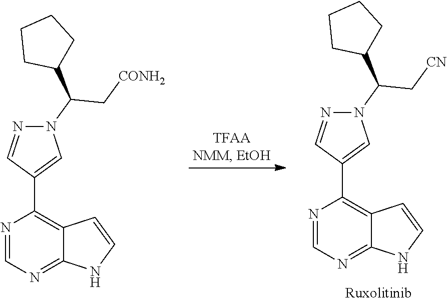 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and ethyl acetate (8 V) were charged into RBF at RT under nitrogen atmosphere, then 4-Methylmorpholine (NMM, 5.0 eq.,) was charged into the reaction mass and stirred for 10-15 min. The reaction mass was cooled to 10-15° C. and trifluoroacetic anhydride (TFAA, 2-3 m. eq.,) was added slowly under nitrogen atmosphere at below 20° C. The reaction mass was stirred for 1-2 hours at 15-20° C. under nitrogen atmosphere. The reaction mass was quenched with aq. NaHCO₃ solution (10 V) and stirred for 15-20 min. The layers were separated, and the aqueous layer was extracted with ethyl acetate (10 V). The combined both organic layer was washed with aqueous NaHCO₃ solution (10 V), DM-water (2× 10 V) and aqueous saturated NaCl (6 V) solution at RT. The organic layer was treated with charcoal, filtered and the solvent from the filtrate was distilled-off under reduced pressure at below 50° C. until 4.0±0.5 V remains in the RBF. n-Heptane (3.0 V) was added to the reaction mass at 50±5° C. then cooled to 25-30° C. The mass was stirred for 1-2 hours at 25-30° C., filtered and washed with a mixture of ethyl acetate: n-Heptane (2V, 1:1 ratio). The obtained wet material was dried under vacuum to get Ruxolitinib crystalline form G. Yield: 13 to 16 g; Purity: >99%

### Example-12: Preparation and Purification of Ruxolitinib

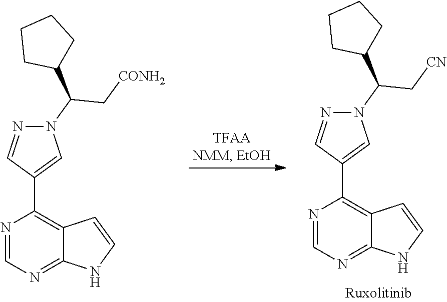 [SMILES: NC(=O)C[C@H](C1CCCC1)n1cc(-c2ncnc3[nH]ccc23)cn1 ; N#CC[C@H](C1CCCC1)n1cc(-c2ncnc3[nH]ccc23)cn1]

(R)-3-(4-(7H-Pyrrolo[2,3-d]pyrimidin-4-yl)-1H-pyrazol-1-yl)-3-cyclopentyl propionamide (20 g) and ethyl acetate (8 V) were charged into RBF at RT under nitrogen atmosphere, then 4-Methylmorpholine (NMM, 5.0 eq.,) was charged into the reaction mass and stirred for 10-15 min. The reaction mass was cooled to 10-15° C. and trifluoroacetic anhydride (TFAA, 2-3 m. eq.,) was added slowly under nitrogen atmosphere at below 20° C. The reaction mass was stirred for 1-2 hours at 15-20° C. under nitrogen atmosphere. The reaction mass was quenched with aq. NaHCO₃ solution (10 V) and stirred for 15-20 min. The layers were separated, and the aqueous layer was extracted with ethyl acetate (10 V). The combined each organic layer was washed with aqueous NaHCO₃ solution (10 V), DM-water (2× 10 V) and aqueous saturated NaCl (6 V) solution at RT. The organic layer was treated with charcoal, filtered and the solvent from the filtrate was distilled-off under reduced pressure at below 50° C. until 4.0±0.5 V remains in the RBF. MTBE (3.0 V) was added to the reaction mass at 50±5° C. then cooled to 25-30° C. The mass was stirred for 1-2 hours at 25-30° C., filtered and washed with a mixture of ethyl acetate: MTBE (2V, 1:1 ratio). The obtained wet material was dried under vacuum to get Ruxolitinib crystalline form G. Yield: 13 to 16 g; Purity: >99%

### Example-13: Preparation and Purification of Ruxolitinib

A slurry of Ruxolitinib phosphate in DCM (10 Vol.) was cooled to 10-15° C. To the mass, water (3 Vol.) was added at 10-15° C. and adjusted the pH to 9-10 using aq. K₂CO₃ solution at 10-15° C. The reaction mass was stirred for 20-30 min at 10-15° C., switched off stirring and allowed the layers to get separated. The aq. layer was extracted with DCM (5-10 Vol.). The combined organic layer was washed with water (10 Vol.) and brine (5-10 vol.). The organic layer was dried over anhydrous sodium sulfate and filtered. The filtrate was concentrated under vacuum until ˜2 Vol remains in RBF. Isopropyl acetate (5 Vol.) was charged into RBF and distilled-off the solvent under vacuum until ˜2 Vol remains in RBF. The mass was heated to 50-55° C. and n-heptane (6 Vol.) was added at 45-55° C. and the mass was stirred for 45 min to 1 h at 50-55° C. The mass was cooled to RT and stirred for 1 to 2 h, filtered and washed with chilled (0-5° C.) isopropyl alcohol (1 V). The wet material was dried under vacuum at 45-50° C. to get Ruxolitinib crystalline form G. Purity: >99% purity.

### Example-14: Preparation and Purification of Ruxolitinib

A slurry of Ruxolitinib phosphate in isopropyl acetate (10-20 Vol.) was cooled to 10-15° C. To the mass, water (3 Vol.) was added at 10-15° C. and adjusted the pH to 9-10 using aq. K₂CO₃ solution at 10-15° C. The reaction mass was stirred for 20-30 min at 10-15° C., switched off stirring and allowed the layers to get separated. The aq. layer was extracted with isopropyl acetate (10-20 Vol.). The combined organic layer was washed with water (10 Vol.) and brine (5-10 vol.). The organic layer was dried over anhydrous sodium sulfate and filtered. The filtrate was concentrated under vacuum until ˜2 Vol remains in RBF. Isopropyl acetate (5 Vol.) was charged into RBF and distilled-off the solvent under vacuum until ˜2 Vol remains in RBF. The mass was heated to 50-55° C. and n-heptane (6 Vol.) was added at 45-55° C. and the mass was stirred for 45 min to 1 h at 50-55° C. The mass was cooled to RT and stirred for 1 to 2 h, filtered and washed with chilled (0-5° C.) isopropyl alcohol (1 V). The wet material was dried under vacuum at 45-50° C. to get Ruxolitinib crystalline form G.

Purity: >99%.

### Example-15: Preparation and Purification of Ruxolitinib

A slurry of Ruxolitinib phosphate in ethyl acetate (10-20 Vol.) was cooled to 10-15° C. To the mass, water (3 Vol.) was added at 10-15° C. and adjusted the pH to 9-10 using aq. K₂CO₃ solution at 10-15° C. The reaction mass was stirred for 20-30 min at 10-15° C., switched off stirring and allowed the layers to get separated. The aq. layer was extracted with ethyl acetate (10-20 Vol.). The combined organic layer was washed with water (10 Vol.) and brine (5-10 vol.). The organic layer was dried over anhydrous sodium sulfate and filtered. The filtrate was concentrated under vacuum until ˜2 Vol remains in RBF. Ethyl acetate (5 Vol.) was charged into RBF and distilled-off the solvent under vacuum until ˜2 Vol remains in RBF. The mass was heated to 50-55° C. and n-heptane (6 Vol.) was added at 45-55° C. and the mass was stirred for 45 min to 1 h at 50-55° C. The mass was cooled to RT and stirred for 1 to 2 h, filtered and washed with chilled (0-5° C.) isopropyl alcohol (1 V). The wet material was dried under vacuum at 45-50° C. to get Ruxolitinib crystalline form G. Purity: >99% purity.

It is to be understood that the present invention is susceptible to modifications, changes and adaptations by those skilled in the art. Such modifications, changes, adaptations are intended to be within the scope of the present invention.

1. An improved process for the preparation of ruxolitinib or a salt thereof, comprising:
a) reacting a compound of formula 2: 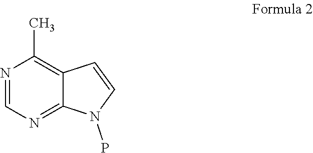
wherein, ‘P’ represents amino protecting group;
with a reagent prepared in-situ from triflic anhydride and DMF in the presence of a suitable solvent to provide a compound of formula 3; 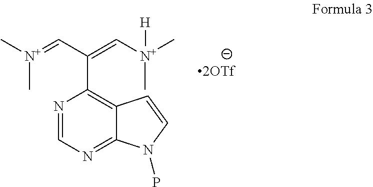
b) contacting the compound of formula 3 with a compound of formula 4 or salt thereof, 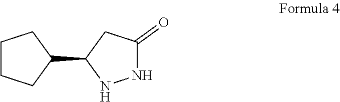 [SMILES: O=C1C[C@H](C2CCCC2)NN1]
to provide a compound of formula 5; 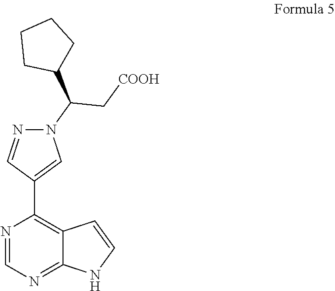 [SMILES: O=C(O)C[C@H](C1CCCC1)n1cc(-c2ncnc3[nH]ccc23)cn1]
c) optionally, purifying the acid intermediate of formula 5 by reacting with dicyclohexylamine (DCHA) in a suitable solvent to provide dicyclohexylamine salt of acid intermediate of formula 5a and 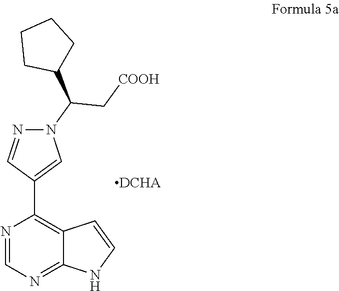 [SMILES: O=C(O)C[C@H](C1CCCC1)n1cc(-c2ncnc3[nH]ccc23)cn1]
further converting into its free base by reacting it with base to provide pure acid intermediate of formula 5;
d) reacting the compound of formula 5 with carbonyldiimidazole (CDI) in a suitable solvent, followed by a reaction with aqueous ammonia to provide a compound of formula 6; 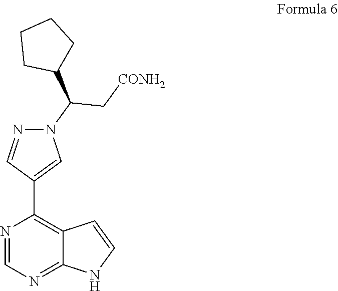 [SMILES: NC(=O)C[C@H](C1CCCC1)n1cc(-c2ncnc3[nH]ccc23)cn1]
e) treating the compound of formula 6 with trifluoroacetic anhydride in the presence of a base and a suitable solvent to provide ruxolitinib; and 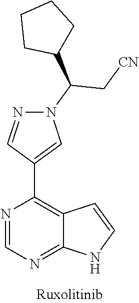 [SMILES: N#CC[C@H](C1CCCC1)n1cc(-c2ncnc3[nH]ccc23)cn1]
f) optionally converting ruxolitinib into its salts thereof.

2. The process as claimed in claim 1, wherein the base is selected from aqueous or alcoholic solution of alkali metal hydroxides, alkaline earth metal hydroxide or organic bases; and the suitable solvent is selected from nitriles, ketones, amides, esters or halogenated solvents.

3. An improved process for the preparation of intermediate compound of formula 3, comprising:
reacting compound of formula 2: 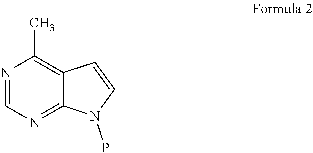
wherein, ‘P’ represents amino protecting group;
with a reagent prepared in-situ from triflic anhydride and DMF in the presence of a suitable solvent to provide a compound of formula 3.

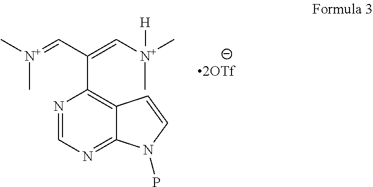

4. The process as claimed in claim 3, wherein the intermediate compound of formula 3 is further converted to ruxolitinib or a salt thereof.

5. An improved process for the preparation of ruxolitinib or a salt thereof, comprising:
a) reacting a compound of formula 5: 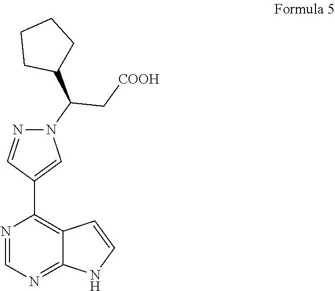 [SMILES: O=C(O)C[C@H](C1CCCC1)n1cc(-c2ncnc3[nH]ccc23)cn1]
with carbonyldiimidazole (CDI) in a suitable solvent, followed by a reaction with aqueous ammonia to provide a compound of formula 6; 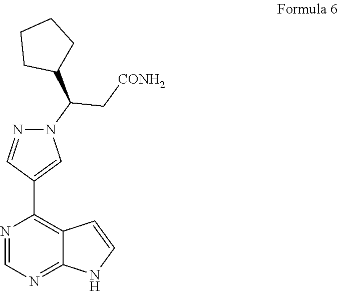 [SMILES: NC(=O)C[C@H](C1CCCC1)n1cc(-c2ncnc3[nH]ccc23)cn1]
b) reacting the compound of formula 6 with trifluoroacetic anhydride in the presence of a base and a suitable solvent to provide ruxolitinib; and 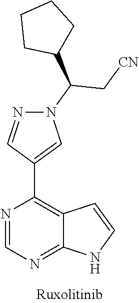 [SMILES: N#CC[C@H](C1CCCC1)n1cc(-c2ncnc3[nH]ccc23)cn1]
c) optionally converting ruxolitinib into its salts thereof

6. An improved process for the preparation of ruxolitinib or a salt thereof, comprising:
a) reacting the compound of formula 6: 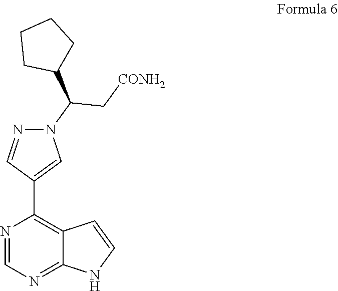 [SMILES: NC(=O)C[C@H](C1CCCC1)n1cc(-c2ncnc3[nH]ccc23)cn1]
with trifluoroacetic anhydride in the presence of a base and a suitable solvent to provide ruxolitinib; and 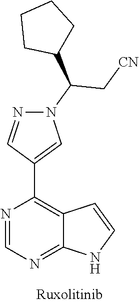 [SMILES: N#CC[C@H](C1CCCC1)n1cc(-c2ncnc3[nH]ccc23)cn1]
b) optionally converting ruxolitinib into its salts thereof.

7-18. (canceled)
